(19) United States
(12) Patent Application Publication
(10) Pub. No.: US 2008/0003688 A1
(43) Pub. Date: Jan. 3, 2008

(54) Method for Measuring the Acidity of Hydrocarbons

(75) Inventors: Philippe Macon, Maneglise (FR); Jacques Maddaluno, Rouen (FR); Alain Bailleul, Allouville Bellefosse (FR); Hassan Oulyadi, Rouen (FR); Jerome Bellanger, Le Havre (FR); Sandrine Renault, Caudebec Les Elbeuf (FR); Martine Beaufour, Maurepas (FR); Mariane Axt, Le Havre (FR)

Correspondence Address: SUGHRUE MION, PLLC, 2100 PENNSYLVANIA AVENUE, N.W., SUITE 800, WASHINGTON, DC, 20037, US

(73) Assignee: Total France, Puteaux (FR)

(21) Appl. No.: 11/794,342

(22) PCT Filed: Dec. 29, 2005
(86) PCT No.: PCT/FR05/03299

§ 371 Date: Jun. 28, 2007

(30) Foreign Application Priority Data
Dec. 30, 2004 (FR) 0414088

(51) Int. Cl. G01N 24/08; G01N 33/28

(52) U.S. Cl. 436/173

(57) ABSTRACT

The invention relates to a novel method for dosing hydroxyl acid function compounds in complex hydrocarbon products by nuclear magnetic resonance of ¹⁹F after reacting hydroxyl acid functions of the product to be analyzed with a fluorinated reactant selected among the β-fluoroolefins.

The present invention relates to a novel method for measuring compounds having acidic hydroxyl functional groups, in particular naphthenic acids and/or phenolic compounds, present in complex hydrocarbon-based products. This measurement is carried out by ¹⁹F nuclear magnetic resonance (NMR) after preparing a fluorinated derivative of the compounds to be measured.

Acid crudes and in particular naphthenic acid crudes are commercially available in large quantities and at prices that are comparatively lower than non-acid crudes. The main drawback of these crudes is their corrosive character which has deleterious effects on the metallic materials of refining plants.

There are currently several methods for evaluating the acidity of petroleum products, among which mention may be made of the determination of the total acid number (TAN) by colorimetry (ASTM D974) or by potentiometry (ASTM D664), or else assaying the carboxylic acids, most of which are naphthenic acids, by measuring the absorption band of carbonyl groups (>C═O) in infrared spectroscopy. Correlating the results from these various known measurements with the corrosive character of the crudes and of the products derived from their refining, has however shown that these measurement methods only give a very imperfect account of the corrosive power of the petroleum products. In other words, the exclusive measurements of carboxylic acids (by IR spectroscopy) or the measurement of all sorts of acid compounds (TAN measured by potentiometry or colorimetry) is not always sufficient to be able to precisely characterize the corrosive character of an acidic crude.

The inability to calculate the corrosive character of a petroleum product by determination of its total acid number (TAN) or of the concentration of naphthenic acids is linked to the inability to individually and selectively detect the various compounds measured globally by the TAN measurement but which are impossible to distinguish in IR spectroscopy. Among these compounds, the phenolic compounds, that is to say the compounds comprising an acidic hydroxyl functional group attached to an aromatic ring, play a particularly important part. Among these compounds naturally present in petroleum products, mention may be made of phenols, cresols (methylphenols) and dimethylphenols. A method of extraction onto a silicone membrane coupled with HPLC analysis (E. F. Laespada, J. L. P. Pavon, B. M. Cordevo, J. Chrom. A, 1999, 852, 395-406) has made it possible to quantify these various phenols in the crude oils and in various oil cuts. This study showed that the phenols are concentrated during the refining process in the cuts having a final boiling point below 150° C.

A better knowledge of the concentration of compounds from this family in the various acidic crude oils and in the distillation cuts obtained from these would probably enable a better calculation of the corrosion properties of these petroleum products.

The Applicant has consequently set the objective of developing an analytical method by nuclear magnetic resonance (NMR) spectroscopy that makes it possible to measure, selectively and independently, both the carboxylic acids, in particular the naphthenic acids, and the phenolic compounds present in the petroleum products, while being free of any sample pretreatment step, such as a liquid/liquid or liquid/solid extraction step. Such a method should make it possible to acquire a better understanding of the corrosion phenomena observed in the refining units, to know the compound or compounds that contribute to the corrosive nature of the petroleum products, to better calculate the corrosive nature of these products, to follow the enrichment of the corrosive compounds in certain cuts or else to be able to compensate for a strong corrosive nature by mixing the acidic products with a suitable amount of equivalent nonacidic products.

Two first series of studies carried out by the Applicant in collaboration with the Fine Organic Chemistry Research Institute (IRCOF) of the University of Rouen (France), and relating to the specific derivatization of phenols and naphthenic acids by reaction with particular reactants and analysis of the derivatives prepared by NMR have unfortunately not succeeded. This was because the alkylation of the phenols and naphthenic acids by ¹³C-labeled alkylating agents followed by the detection of the derivatives prepared by ¹³C NMR proved too expensive and difficult to implement. A second approach consisting in making the phenols and naphthenic acids react with enol ethers (R₁CH═C—O—R₂), known to form, with compounds having acid hydroxyl functional groups, acetals that are easy to detect by proton NMR proved to suffer from a lack of sensitivity.

Within the context of these studies in collaboration with IRCOF, a third final study, crowned with success, consisted in using fluoroolefins for the derivatization of the phenols and naphthenic acids and of detecting the fluorinated derivatives that were formed by fluorine (¹⁹F) NMR. The nucleus ¹⁹F, which has a natural abundance of 100% and a gyromagnetic ratio close to that of the proton, may be detected by NMR with a sensitivity of around 83% of that of the proton. In addition to this high sensitivity, this nucleus has a wide range of chemical shifts extending over about 250 ppm which gives a good resolution between signals corresponding to molecules that are structurally similar. Finally, another very important advantage of the use of ¹⁹F in NMR is the absence of this element in crude oils and oil cuts. The result of this is that all the resonance peaks recorded may in principle be attributed to the introduction of the fluorinated reactant and to the various products formed by reaction of this reactant with the components of the petroleum product and/or with the ingredients of the reaction solution.

Consequently, one subject of the invention is a method for measuring compounds having acidic hydroxyl functional groups in complex hydrocarbon-based products by ¹⁹F NMR after reaction of the acidic hydroxyl functional groups of the product to be analyzed with a fluorinated reactant chosen from β-fluoroolefins, that is to say olefins comprising at least one fluorine atom at the beta position of the double bond.

Another subject of the invention is the use of a β-fluoroolefin as a fluorinated reactant for measuring compounds having acidic hydroxyl functional groups by ¹⁹F NMR.

The method for measuring compounds having acidic hydroxyl functional groups in complex hydrocarbon-based products by ¹⁹F NMR, in particular comprises the steps (a) to (d) below:
(a) preparing a reaction solution by mixing,
   a sample of the complex hydrocarbon-based product containing the compounds having acid hydroxyl functional groups to be measured;
   a β-fluoroolefin capable of reacting with the acidic hydroxyl functional group of the compounds to be measured, so as to form the corresponding acetal;
   a strong organic acid, used as a catalyst for this derivatization reaction;
   a fluorinated internal standard; and
   an organic solvent capable of dissolving the above compounds,
(b) reacting the β-fluoroolefin with the compounds having acidic hydroxyl functional groups to be measured over a sufficient period to convert all the compounds having acidic hydroxyl groups to be measured into their acetal derivative;
(c) acquiring a high-resolution ¹⁹F NMR spectrum of the reaction solution; and
(d) processing the recorded spectrum and calculation of the concentration of compounds having acidic hydroxyl groups in the complex hydrocarbon-based product.

The method of the present invention enables the direct analysis of petroleum product samples, without a prior purification or extraction step. It is a very simple method having a single reaction step, said step moreover possibly being carried out directly in the tube being used to acquire the NMR spectrum. This method makes it possible, unlike the analysis methods of the prior art, to separately detect and measure the phenolic compounds and the naphthenic acids with sensitivities equivalent to or greater than those of the known measuring methods.

The complex hydrocarbon-based product whose content of carboxylic acids, which are predominantly naphthenic acids and/or phenolic compounds, has to be measured, may in principle be any product, of natural or synthetic origin, optionally having undergone one or more treatment or refining steps, on the condition that it is soluble in the analysis solvent. The method according to the invention is particularly useful for analyzing acidic crude oils and the refining products derived from these, in particular petrol, kerosene or diesel cuts obtained by distillation at atmospheric pressure of acidic crudes or by distillation at reduced pressure of atmospheric residues.

The term “carboxylic acids” is understood to mean in the present invention all compounds comprising one or more carboxyl (—COOH) functional groups attached to an aliphatic chain or to an aromatic ring. The aliphatic chain may be a linear or branched chain, or a cycloaliphatic group comprising one or more condensed or uncondensed, saturated rings.

The term “naphthenic acids” as it is used in the present invention covers all hydrocarbon-based compounds comprising one or more condensed saturated rings and one or more carboxylic acid functional groups attached to these saturated rings, either directly or via an alkylene group.

The term “phenolic compounds or phenols” is understood in the present invention to mean all compounds comprising one or more hydroxyl functional groups attached to an aromatic or heteroaromatic, monocyclic or polycyclic ring, optionally bearing one or more alkyl substituents.

The β-fluoroolefin used in the method of the present invention for preparing a fluorinated derivative that can be detected by ¹⁹F NMR is preferably a compound corresponding to the formula (I) below:
CFₙR¹₍₃₋ₙ₎—CH═CH—X—R² (I)
in which:
X represents an oxygen or sulfur atom, or an —NR³— group, where R³ represents a C₁₋₄ alkyl group;
n is equal to 1, 2 or 3; and
R¹ is a hydrogen atom or a C₁₋₆ alkyl group;
R² represents a C₁₋₈ alkyl group having a linear or branched chain, optionally comprising, in the alkyl chain or in a substituent borne by the alkyl chain, one or more atoms having lone pairs of electrons, preferably chosen from oxygen, sulfur or nitrogen atoms.
X preferably represents an oxygen atom.

The value of n is preferably equal to 2 or 3. This is because the area of the resonant signal, that is to say the sensitivity of the NMR method, is directly proportional to the number of fluorine atoms present in the derivatization reactant. Compounds of formula (I) where n equals 3 are consequently most particularly preferred.

The derivatization reaction of the compounds to be measured by the β-fluoroolefin in the presence of an acid catalyst is carried out by nucleophilic attack of the acidic hydroxyl functional group of the compound to be measured (R′OH) according to the following mechanism:

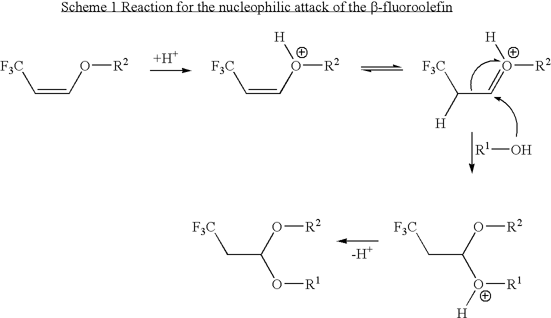

The atom having a lone pair of electrons is preferably the oxygen atom of an ether-oxide group in the R₂ alkyl chain. This ether group is preferably separated from the first —O— group by two carbon atoms.

The Applicant has obtained a suitable reactivity by using, as the β-fluoroolefin of formula (I), 1-(2-ethoxyethoxy)-3,3,3-trifluoropropene, and in particular the Z isomer of this. 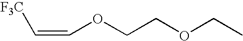
1-(2-ethoxyethoxy)-3,3,3-trifluoropropene, Z isomer

The synthesis and characterization of this compound has been carried out following the results of Hong et al. published in J Chem. Soc. Chem. Commun. 1996, 57-58.

In order to be able to be certain to derive and measure all the phenolic compounds and/or naphthenic acids present in the petroleum products, the β-fluoroolefin must of course be added to the reaction solution in excess relative to the amount of these compounds that are expected to be found in the sample to be measured. This amount generally does not exceed a few hundreds of milligrams of phenolic compounds per liter of petroleum product (see E. F. Laespada, J. L. Pavon, B. M. Cordevo, J. Chrom. A, 1999 852, 395-406) and about 3% by weight of naphthenic acids.

The Applicant has, in addition, observed that the β-fluoroolefin does not only react with the naphthenic acids and/or phenolic compounds of the petroleum product but also with itself to form an acetal of formula: 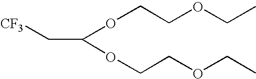
or with the acid used as a catalyst for the reaction, for example camphorsulfonic acid, to form an acetal of formula: 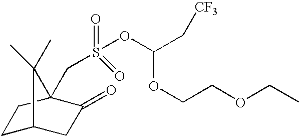
secondary reactions that consume a certain molar fraction of the derivatization reactant.

The strong acid used as a catalyst for the derivatization reaction must be a relatively hydrophobic organic acid that is soluble in the hydrocarbon-based reaction medium. The Applicant has tested several organic acids among which mention may be made of acetic acid, trifluoroacetic acid, p-toluenesulfonic acid and camphorsulfonic acid. It is the latter acid which has given the best results in terms of reactivity and solubility in the reaction medium and consequently camphorsulfonic acid will preferably be used as a catalyst for the acetalization reaction of the method of the present invention.

The catalyst is preferably added to the reaction solution in an amount of 2 to 10 mol %, relative to the amount of derivatization reactant (β-fluoroolefin). At concentrations below 2 mol %, the acetalization reaction becomes too slow which makes the measuring time unacceptably long. For concentrations exceeding 10 mol % relative to the β-fluoroolefin, secondary reactions leading to background peaks in the integration area of the NMR spectrum become too large and are liable to distort the measurement results.

The fluorinated internal standard is a compound of known structure, added at a known concentration, which does not participate in the reaction but is used to establish the relationship between the signals (the areas of various resonance peaks) and the concentrations of the fluorinated compounds corresponding to these peaks. For reasons of ease of implementation of the method, the internal standard is preferably added to the initial reaction mixture before the derivatization reaction of the compounds to be measured, but adding an internal standard after completion of the reaction, immediately before the step of acquiring the spectrum, could of course also be envisaged.

The internal standard must be a fluorinated compound that is pure, stable, nonvolatile and which gives a signal that is perfectly distinct from the other signals of the spectrum.

Mention may be made of trifluorotoluene (TFT) as an example of an internal standard that can be used for the present invention. This compound has excellent chemical stability under the reaction conditions, has a sufficiently high boiling point (T_{b}=102° C.) to prevent its evaporation in the course of the reaction and the spectrum acquisition step, and has, in addition, a chemical shift close to that of the expected acetals. TFT is referenced as having a chemical shift δ of 99.09 ppm relative to the signal of hexafluorobenzene (C₆F₆). It is however possible to use other fluorinated compounds that combine the properties mentioned above.

The various compounds described above (sample, β-fluoroolefin, catalyst, internal standard) must be dissolved in a suitable organic solvent. This solvent, which must not disturb the ¹⁹F NMR spectrum by resonance of its own atoms, is generally a deuterated solvent. In principle it will be possible to use any deuterated organic solvent that is sufficiently hydrophobic to dissolve the sample to be measured, and also the reactants, catalysts and standards used, and which is preferably perfectly inert with regard to the derivatization reaction. The Applicant in particular prefers to use deuterated chloroform (CDCl₃), as it is a common NMR solvent which has properties close to that of the dichloromethane proposed for the formation of acetals by reaction of hydroxylated compounds with trifluoropropene ethers (F. Hong and C. M. Hu, J. Chem. Soc. Chem. Commun. 1996, 57-58).

After mixing the various components of the reaction solution from step (a), the derivatization reaction is left to take place over a sufficient time to convert all the phenolic compounds and/or naphthenic acids to the corresponding fluorinated acetals. This time may be shortened by gentle heating of the reaction solution, preferably at a temperature between 30 and 50° C., and in particular between 35 and 45° C.

In one preferred embodiment of the method of the invention, step (a) of preparing the reaction solution and the derivatization reaction (step (b)) are carried out directly in the NMR tube used to acquire the ¹⁹F NMR spectrum (step (c)). It is then possible to follow the progression of the acetalization reaction by producing a series of spectra at regular intervals. The reaction is then considered to be completed when the area of the resonance peaks corresponding to the acetals of naphthenic acids and/or of phenolic compounds no longer increases over time. The reaction time necessary to achieve a complete reaction is generally between 20 minutes and 4 hours, preferably between 30 and 150 minutes.

The quantitative fluorine NMR used for the method of the present invention is high-resolution NMR, that is to say pulsed nuclear magnetic resonance carried out with the following acquisition parameters:

Spectrometer: 400 MHz BRUKER

Probe: 5 mm QNP

Nucleus: ¹⁹F

Pulse sequence: 1D sequence with ¹H decoupling during acquisition

Decoupling program: Waltz 16

Number of scans: 32

Spectral window: 40-50 ppm

p1: 14.5 us

t10: 0.0 dB

d1: 10 s

1b: 1.0 Hz

td: 64 K

si: 64 K

The present invention is illustrated by the following examples that describe the synthesis and characterization of the fluorinated reactant (Example 1), the validation of the measurement method on model compounds (Example 2), the application of the measurement method for determining the total acid number of oil cuts (Example 3) and the possibility of separate detection of phenolic compounds and naphthenic acids (Example 4).

### EXAMPLE 1

### Synthesis and Characterization of 1, (2-ethoxyethoxy)-3,3,3-trifluoropropene

7.5 g (0.134 mol) of potassium hydroxide were dissolved in 1.8 ml (0.1 mol) of distilled water. Next, 15 ml (13.95 g, 0.155 mol) of 2-ethoxyethanol were added, then 7.5 g (about 5 ml, 0.043 mol) of 2-bromo-3,3,3-trifluoropropene were slowly added. During this first step, the mixture was cooled, if necessary, with an ice bath to keep it at room temperature. The reactor was equipped with an acetone condenser to prevent the evaporation of the bromotrifluoropropene (T_{b}=33° C.). After reacting for 150 minutes, the reaction medium was washed four times with 15 ml or water. During the fourth wash, 15 ml of diethyl ether was added. The ether phase was separated and subjected to a vacuum evaporation, the residue obtained was distilled under vacuum (about 15 mm Hg) using a heat gun at a temperature between 55 and 60° C. In this way 4.66 g of 1-(2-ethoxyethoxy)-3,3,3-trifluoropropene was obtained which corresponded to a yield of 59%.

¹H NMR (CDCl₃, 400 MHz), δ (ppm) TMS: 6.44 (1H, d, ³J=7.0 Hz), 4.62 (1H, dq, ³J=8.2 Hz, ³J=7.3 Hz), 4.07 (2H, dd, ³J=3.2 Hz, ³J=4.8 Hz), 3.65 (2H, dd, ³J=2.9, ³J=4.4 Hz), 3.54 (2H, q, ³J=7.1 Hz), 1.21 (3H, t, ³J=7.1 Hz)

¹³C NMR (CDCl₃, 75 MHz), δ (ppm) TMS: 154.2 (q, ³J=5.6 Hz); 123.4 (q, ¹J=268.6 Hz); 94.2 (q, ²J=34.6 Hz); 73.8; 69.5; 66.9; 15.0.

### EXAMPLE 2

### Validation of the Method by Verification of the Quantitative Yield of the Derivatization Reaction

A reaction solution was prepared containing a known amount of phenol and of cyclohexanecarboxylic acid (example of naphthenic acid) by mixing, in an NMR tube, the following ingredients:

65 μmol (12 mg) of 1-(2-ethoxyethoxy)-3,3,3-trifluoropropene (RCF₃);

5 μmol of phenol;

5 μmol of cyclohexanecarboxylic acid;

5 μmol of trifluorotoluene (internal standard);

camphorsulfonic acid (CSA), 10 mol % relative to RCF₃; and

CDCl₃ up to a total volume of around 500 μl.

An ¹⁹F NMR spectrum with proton decoupling was acquired by using the NMR parameters indicated above, firstly at intervals of 10 minutes, over one hour, then at intervals of half an hour, for an additional 3 hours. The temperature of the NMR probe was set at 40° C.

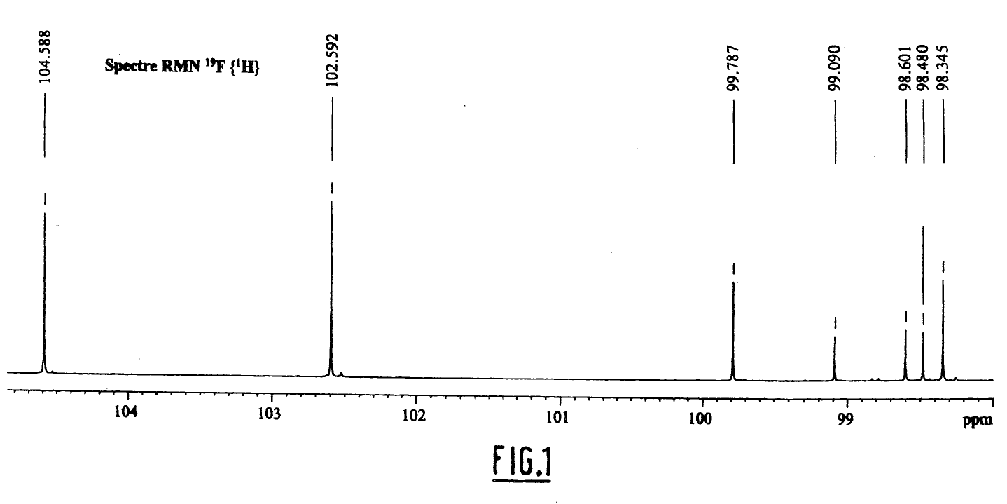

FIG. 1 shows such a spectrum recorded at the end of 2 hours. This spectrum has seven resonance signals which may be attributed respectively to the following compounds:

TABLE 1
Attribution of the referenced ¹⁹F NMR resonance signals
relative to TFT (δ = 99.09 ppm)

| ¹⁹F δ (ppm) | Attribution |
|---|---|
| 104.60 | Z isomer of RCF₃ |
| 102.60 | E isomer of RCF₃ |
| 99.74 | Product of the RCF₃ + camphorsulfonic |
| | acid reaction |
| 99.09 | Trifluorotoluene (internal standard) |
| 98.50-98.85 | Acetal of cyclohexanecarboxylic acid |
| 98.44-98.50 | Acetal of phenol |
| 98.34 | Product of the reaction of RCF₃ with itself |

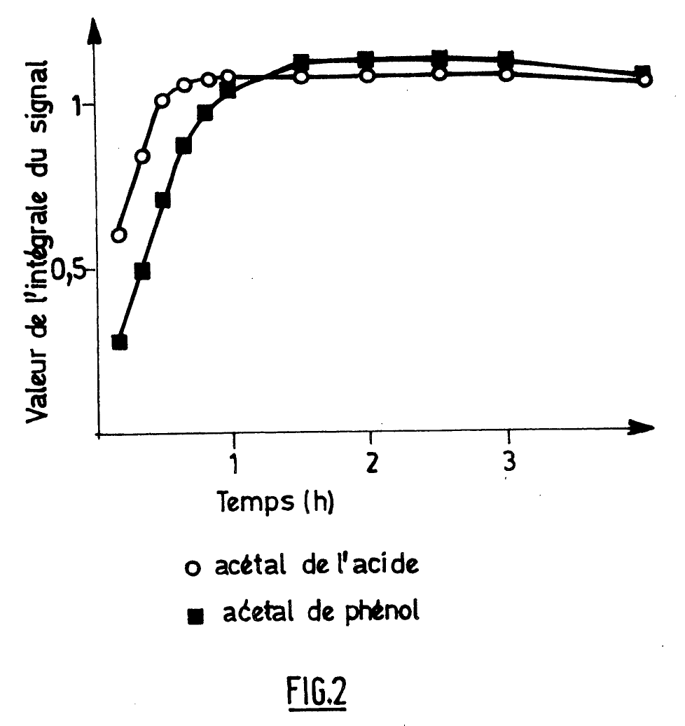

FIG. 2 shows the change over time of the area of the signals corresponding respectively to the acetal of cyclohexanecarboxylic acid (δ=98.60 ppm) and to the acetal of phenol (δ=98.48 ppm). The area of the signals have been standardized relative to that of the signal of the internal standard whose value has been set to 1.

This figure shows that the kinetics of the derivatization reaction of cyclohexanecarboxylic acid is faster than that of the derivatization of phenol. Indeed, the area of the signal corresponding to the acetal of cyclohexanecarboxylic acid no longer increases after about 50 minutes, whereas the curve corresponding to the acetal of phenol only reaches a plateau at the end of about one and a half hours. The value of the plateau achieved by the two curves is slightly greater than 1. This deviation relative to the theoretical value of 1 is due to the presence of low intensity signals of the same chemical shift.

The results of these prior tests indicate that the derivatization reaction of naphthenic acids and/or of phenols by RCF₃ is quantitative in less than 2 hours, that the fluorine NMR allows this reaction to be followed perfectly. This method should consequently make it possible to measure the phenol compounds and naphthenic acids in the oil cuts containing them.

### EXAMPLE 3

### Measurement of Phenolic Compounds and Naphthenic Acids in Oil Cuts

In this example fluorine NMR was used to measure the phenols and naphthenic acids in the following three oil cuts having various acidities:
a slightly acidic Basrah light cut;
a slightly more acidic Forcados cut; and
a Captain cut having a high acidity.

In order to do this, a reaction solution according to the invention was prepared for each of these cuts, and also a “blank” or “negative control” having a composition identical to that of the reaction solution apart from the fact that the volume of the oil cut is replaced by an identical volume of solvent (see Table 2). The blank will be used to acquire a spectrum showing all the low-intensity background peaks. This spectrum will then be subtracted from each of the spectra obtained for the three oil cuts.

TABLE 2
Composition of the reaction solution and the negative control

| | |
|---|---|
| Reaction | Negative |
| solution | control |

| Oil cut | 120 | μl | — | |
|---|---|---|---|---|
| CDCl₃ solvent | 54 | μl | 174 | μl |
| 1-(2-ethoxyethoxy)- | 100 | μl | 100 | μl |
| 3,3,3-trifluoropropene | | | | |
| (RCF₃) at 120 mg/ml | | | | |
| Camphorsulfonic acid | 190 | μl | 190 | μl |
| at 4 mg/l (catalyst) | | | | |
| Trifluorotoluene at | 36.5 | μl | 36.5 | μl |
| 20 mg/ml (internal standard) | | | | |

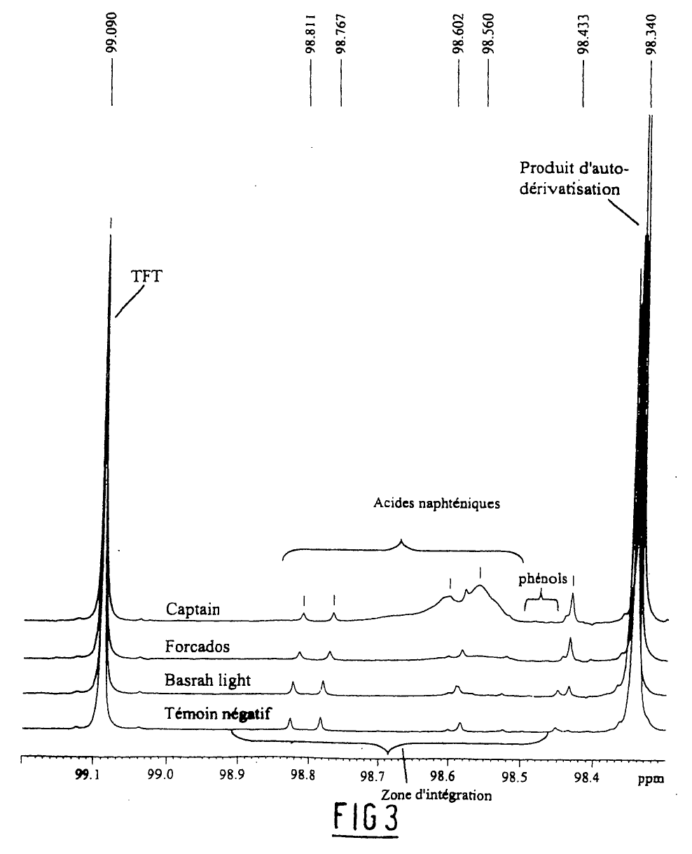

The three reaction solutions were reacted for 2 hours at 40° C. and the negative control was exposed to the same conditions for 2 hours. Next the ¹⁹F NMR spectrum with proton decoupling was acquired by using the NMR parameters indicated in Example 1, for the negative control and for each of the three reaction solutions. FIG. 3 shows the section between 98.3 ppm and 99.2 ppm of the ¹⁹F NMR spectra obtained for the three oil cuts analyzed and also the spectrum of the negative control.

The value of the sum of the integrals of the signals corresponding to the acetals of phenols and naphthenic acids (integration area of FIG. 3) make it possible to calculate a total acid number (TAN_{calculated}), expressed in mg of KOH per g of oil cut, according to the following formula:

$${TAN}_{calculated} = \frac{( {I}_{2} - {I}_{1} )}{{I}_{IS}} \times \frac{{n}_{IS} \times {M}_{KOH}}{{m}_{cut}}$$

where
I₂ is the integral value of the oil cut spectrum over the integration area corresponding to the phenols and naphthenic acids (98.91 ppm-98.46 ppm);
I₁ is the integral value of the negative control spectrum over the same integration area;
I_{IS} is the integral value of the internal standard (TFT) signal, namely 1;
n_{IS} is the number of moles of internal standard introduced into the reaction solution, in this case 5×10⁻⁶ mol;
M_{KOH} is the molar mass of KOH, expressed in mg/mol, namely 56105.6; and
m_{cut} is the mass of the cut introduced into the reaction solution, expressed in grams.

At the same time, the value of the total acid number was determined by colorimetry in accordance with the standard ASTM D974 (TAN_{colorimetry}) and these values were compared to those of the TAN calculated from the ¹⁹F NMR spectra. The results are given in Table 3 below.

TABLE 3
Comparison of TAN_{colorimetry} and TAN_{calculated} (¹⁹F NMR)

| | Oil cut | | |
|---|---|---|---|
| | Basrah light | Forcados | Captain |
| TAN_{colorimetry} | <0.1 | 0.3 | 2.6 |
| TAN_{calculated} | 0.2 ± 1.5% | 0.4 ± 4.1% | 2.7 ± 0.9% |
| (±standard | | | |
| deviation) | | | |

It was observed that for the acidic and slightly acidic oil cuts, the results obtained by the method according to the invention are in agreement with those obtained according to the calorimetric method of the prior art.

### EXAMPLE 4

### Differentiation of the Phenols and Naphthenic Acids

The derivatization of a series of known phenolic compounds, liable to be present in oil cuts, was carried out by subjecting them to reaction conditions similar to those from Examples 2 and 3, then tracing the ¹⁹F NMR spectra of the acetals obtained. Table 4 below shows the chemical shifts of the acetals of the main phenolic compounds tested.

TABLE 4
Chemical shift of the acetals of the main phenolic
compounds present in the petroleum products, referenced
relative to TFT (δ = 99.09 ppm)

| Phenolic compound | ¹⁹F δ of the acetal (in ppm) |
|---|---|
| phenol | 98.48 |
| o-methylphenol | 98.48 |
| m-methylphenol | 98.43 |
| 2,4-dimethylphenol | 98.49 |
| 2,6-dimethylphenol | 98.74 |
| 3,4-dimethylphenol | 98.45 |

It was observed that the signals of the derivatives of the phenolic compounds were in the main located between 98.4 and 98.5 ppm. Only 2,6-dimethylphenol (δ=98.74) was the exception to this rule.

The values of the chemical shifts above show that measuring by ¹⁹F NMR makes it possible to separately detect the phenols (resonance peaks between 98.4 and 98.5 ppm) and the naphthenic acids which give signals between 98.5 and 98.9 ppm (see FIG. 3).

1. A method for measuring compounds having acidic hydroxyl functional groups in complex hydrocarbon-based products by ¹⁹F NMR after reaction of the acidic hydroxyl functional groups of the product to be analyzed with a fluorinated reactant chosen from β-fluoroolefins.

2. The measuring method as claimed in claim 1, characterized in that the β-fluoroolefin corresponds to the formula (I) below:
CFₙR¹₍₃₋ₙ₎—CH═CH—X—R² (I)
in which:
X represents an oxygen or sulfur atom, or a —NR³— group, where R³ represents a C₁₋₄ alkyl group;
n is equal to 1, 2 or 3; and
R¹ is a hydrogen atom or a C₁₋₆ alkyl group;
R² represents a C₁₋₈ alkyl group having a linear or branched chain, optionally comprising, in the alkyl chain or in a substituent borne by the alkyl chain, one or more atoms having lone pairs of electrons, preferably chosen from oxygen, sulfur or nitrogen atoms.

3. The method as claimed in claim 2, characterized in that X represents an oxygen atom.

4. The method as claimed in claim 2, characterized in that R² represents a C₁₋₈ alkyl group comprising at least one ether bond (—O—).

5. The method as claimed in claim 2, characterized in that n is equal to 2 or 3, preferably 3.

6. The method as claimed in claim 1, characterized in that the β-fluoroolefin is 1-(2-ethoxyethoxy)-3,3,3-trifluoropropene, preferably the Z isomer of this compound.

7. The method for measuring compounds having acidic hydroxyl functional groups in complex hydrocarbon-based products by ¹⁹F NMR as claimed in any one of the preceding claims, characterized in that it comprises the steps (a) to (d) below:
(a) preparing a reaction solution by mixing,
a sample of the complex hydrocarbon-based product containing the compounds having acid hydroxyl functional groups to be measured;
a β-fluoroolefin capable of reacting with the acidic hydroxyl functional group of the compounds to be measured, so as to form the corresponding acetal;
a strong organic acid, used as a catalyst for this derivatization reaction;
a fluorinated internal standard; and
an organic solvent capable of dissolving the above compounds,
(b) reacting the β-fluoroolefin with the compounds having acidic hydroxyl functional groups to be measured over a sufficient period to convert all the compounds having acidic hydroxyl groups to be measured into their acetal derivative;
(c) acquiring a high-resolution ¹⁹F NMR spectrum of the reaction solution; and
(d) processing the recorded spectrum and calculation of the concentration of compounds having acidic hydroxyl groups in the complex hydrocarbon-based product analyzed.

8. The method as claimed in claim 7, characterized in that the strong organic acid, used as a catalyst, is camphorsulfonic acid.

9. The method as claimed in claim 8, characterized in that the amount of strong organic acid, used as a catalyst, is between 2 and 10 mol %, relative to the amount of β-fluoroolefin used.

10. The method as claimed in claim 7, characterized in that deuterated chloroform (CDCl₃) is used as an organic solvent.

11. The method as claimed in claim 7, characterized in that step (b) comprises heating the reaction solution at a temperature between 30 and 50° C., preferably between 35 and 45° C.

12. The method as claimed in claim 7, characterized in that the length of step (b) is between 20 minutes and 4 hours, preferably between 30 and 150 minutes.

13. The method as claimed in claim 7, characterized in that step (a) for preparing the reaction solution and step (b) of the derivatization reaction are carried out in the NMR tube used to record the ¹⁹F NMR spectrum (step (c)).

14. The use of a β-fluoroolefin as a fluorinated reactant for measuring compounds having acidic hydroxyl functional groups by ¹⁹F NMR.
